(19) United States
(12) Patent Application Publication
(10) Pub. No.: US 2026/0256970 A1
(43) Pub. Date: Sep. 3, 2026

(54) Enclosure for Disinfecting a Medical Device

(71) Applicant: GERMITEC, Bordeaux (FR)

(72) Inventors: Fanny POULON, Bures Sur Yvette (FR); Aimrane HAMDOU, Bordeaux (FR)

(21) Appl. No.: 19/551,253

(22) Filed: Feb. 26, 2026

(30) Foreign Application Priority Data
Feb. 28, 2025 (FR) FR2502034

(51) Int. Cl. A61L 2/10; A61L 103/15

(52) U.S. Cl. CPC A61L 2/10; A61L 2103/15; A61L 2202/11

(57) ABSTRACT

Enclosure for disinfecting a medical device The present invention relates to an enclosure (10) for disinfecting a medical instrument (12), the enclosure (10) comprising: - a first casing (16) defining a first housing (20) closed by a first partition (22), and - a second casing (18) defining a second housing (32) closed by a second partition (34). The second casing (18) is movable relative to the first casing (16) between an open position and a closed position. In the closed position, the first partition (22) and the second partition (34) define between them a closed disinfection volume. The enclosure (10) further comprising a UV radiation generation device configured to generate UV radiation in the disinfection volume, the UV radiation generation device being arranged in at least one of the first housing (20) and the second housing. Figure for the abstract: FIG. 1

### BACKGROUND

The present invention relates to an enclosure for disinfecting a medical instrument.

In order to avoid any contamination, a medical instrument should be disinfected between two uses.

For this purpose, it is known to disinfect medical devices instruments in enclosures for disinfecting defining a closed disinfection volume. Such enclosures comprise, for example, a UV (Ultraviolet) radiation generation device allowing, notably, to carry out disinfection of these medical instruments, and more particularly a high-level disinfection.

Certain medical devices, such as medical probes for example, typically comprise at least one active part and generally at least one connection part in the form of a cable.

It is known to suspend these medical instruments in the enclosure for disinfecting, for example using a suspension stand comprising for example a knob or a ring able to hold a portion of the connection part.

However, such enclosures are not suited to medical instruments without a connection cable, such as portable probes for example.

Furthermore, such enclosures are poorly suited to the disinfection of large and/or asymmetric probes.

Indeed, for such probes, there is a risk that the probe touches one of the walls of the enclosure, thus creating a non-disinfected area, commonly called a "cold spot".

Furthermore, such contact between the probe, and the walls can lead to a risk of damage to the UV sources arranged on the walls.

One of the aims of the invention is therefore to remedy these drawbacks, and in particular to propose a robust enclosure for disinfecting able to ensure high-level disinfection of a medical instrument, while being compatible with a wide variety of medical instruments.

To this end, the invention has as its object an enclosure for disinfecting a medical instrument, the enclosure comprising:

a first casing defining a first housing closed by a first partition, and

a second casing defining a second housing closed by a second partition;

the second casing being movable relative to the first casing between an open position and a closed position,

in the closed position, the first partition and the second partition defining between them a closed disinfection volume;

the enclosure further comprising a UV radiation generation device configured to generate UV radiation in the disinfection volume, the UV radiation generation device being arranged in at least one of the first housing and the second housing.

Indeed, by means of the partitions defining a support for the medical instrument, such an enclosure allows to disinfect a wide range of instruments, of varied sizes and shapes, whether or not they are equipped with cables.

In addition, the partitions prevent the medical instrument from touching the walls of the enclosure, which allows to limit the creation of non-disinfected areas and thus ensure satisfactory disinfection.

Furthermore, the partitions allow to protect the UV radiation generation device, and thus prevent it from being damaged.

According to other advantageous aspects of the invention, the enclosure for disinfecting comprises one or more of the following features, taken alone or according to all technically possible combinations:

At least one of the first partition and the second partition comprises a central portion able to define a receiving housing for the medical instrument.

One of the first and the second partitions is transparent to UV radiation.

At least one of the first and the second partitions has a UV transmittance greater than 30%, and preferably greater than 50%.

At least one of the first and the second partition is made of quartz.

The enclosure further comprises a hinge arranged between the first casing and the second casing and allows the second casing to move relative to the first casing.

The disinfection volume is cylindrical.

The UV radiation generation device comprises a plurality of UV sources arranged in at least one of the first housing and the second housing.

The first casing comprises at least one additional electronic component arranged in the first housing.

The at least one electronic component is an identification unit or a disinfection control unit.

The invention will appear more clearly upon reading the following description, given solely by way of non-limiting example, and made with reference to the drawings in which:

### BRIEF DESCRIPTION OF THE DRAWINGS

FIG. 1 is a schematic perspective representation of an enclosure for disinfecting according to the invention, the enclosure being in the open position, and

FIG. 2 is a schematic sectional representation of the enclosure for disinfecting of FIG. 1 in the closed position.

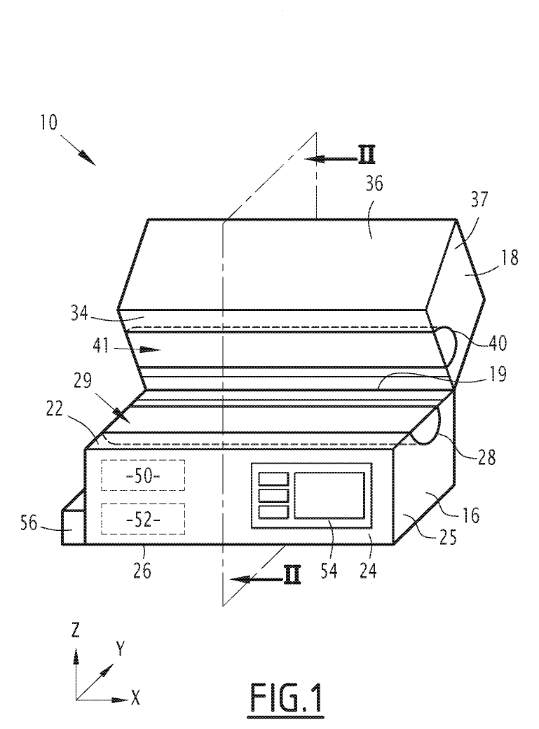

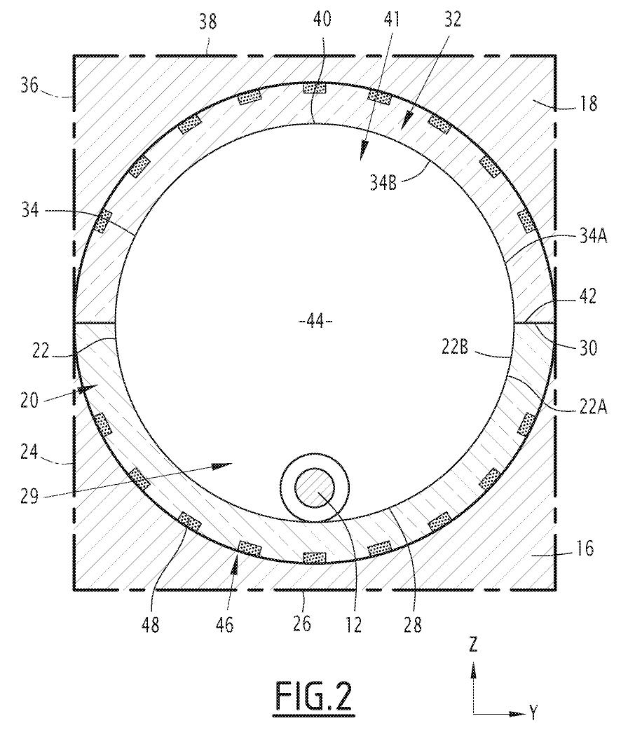

FIGS. 1 and 2 illustrate an example of an enclosure for disinfecting 10 according to the invention.

### DETAILED DESCRIPTION

The enclosure for disinfecting 10 is for example configured to disinfect an entire medical instrument 12.

By disinfecting the entire medical instrument 12, it is meant disinfection of all parts of the medical instrument 12 in the enclosure for disinfecting 10.

The medical instrument 12 is for example a medical probe, notably an ultrasound medical probe, for example an endocavity probe or a transesophageal probe. The medical probe is, for example, a linear probe or a convex probe. The medical probe is for example a pediatric or adult probe.

The medical instrument 12 comprises, for example, an active part. The active part is, for example, intended to be in direct or indirect contact with the patient during its use, depending on the medical application.

The active part of the medical instrument 12 has for example a length between 10 cm and 40 cm.

The medical instrument 12 is for example a wireless medical instrument.

By "wireless", it is meant that the medical instrument does not comprise a connection part in the form of a cable for example.

In particular, the medical instrument 12 comprises, for example, a battery and/or a wireless communication module.

Alternatively, the medical instrument 12 further comprises a connection part and, for example, also a connector. The connection part is for example a connection cable connecting the active part to the connector. The connector is for example configured to be connected to a measurement and/or display unit, notably so as to collect and/or display information captured by the active part.

The enclosure 10 comprises a first casing 16 and a second casing 18.

The second casing 18 is movable relative to the first casing 16 between an open position, illustrated in FIG. 1, and a closed position, illustrated in FIG. 2.

In the particular example illustrated in the Figures, the enclosure 10 has, in the closed position, the shape of a rectangular parallelepiped, in particular, defining an associated orthonormal reference frame comprising a longitudinal axis X, a transverse axis Y and a vertical axis Z.

For example, the enclosure 10 comprises notably, a hinge 19 arranged between the first casing 16 and the second casing 18 and allowing the second casing 18 to move relative to the first casing 16.

In particular, the second casing 18 is for example pivotable according to a pivot axis parallel to the longitudinal axis X relative to the first casing 16 between the open position and the closed position.

The first casing 16 defines a first housing 20 closed by a first partition 22.

Preferably, the first casing 16 comprises at least one wall 24, 25, 26.

For example, the first casing 16 comprises a plurality of walls 24, 25, 26 defining between them the first housing 20.

In the embodiment of the invention illustrated in FIGS. 1 and 2, the plurality of walls of the first casing 16 comprises for example two longitudinal walls 24 extending perpendicular to the transverse axis Y, two transverse walls 25 extending perpendicular to the longitudinal axis X and each connecting the two longitudinal walls 24 according to the transverse axis Y, and a bottom 26 extending perpendicular to the vertical axis Z and connecting the two longitudinal walls 24 according to the transverse axis Y and the two transverse walls 25 according to the longitudinal axis X.

Each of the walls 24, 25, 26 is for example a plate, notably substantially rectangular.

In one alternative, not illustrated, the first casing 16 comprises a single wall delimiting the first housing 20, which is for example semi-cylindrical or semi-spherical.

The first partition 22 closes the first housing 20.

In other words, the first housing 20 is a closed volume.

More precisely, the first housing 20 is delimited between the wall or walls 24, 25, 26 of the first casing 16 and the first partition 22.

In the particular illustrated embodiment, the first partition 22 extends notably facing the bottom 26.

Preferably, the first partition 22 is fixed to each of the two longitudinal walls 24 and the two transverse walls 25, and notably to the upper edge of each of these four walls 24, 25 as is visible in the Figures.

By upper edge, it is meant here the edge of each of these four walls 24, 25 opposite the bottom 26.

As is visible in the Figures, the first partition 22 comprises an internal face 22A, in particular oriented toward the first housing 20, and an external face 22B opposite the internal face 22A.

The first partition 22 is preferably a plate.

In particular, the thickness of the first partition 22, in other words, the distance according to the vertical axis Z between the two faces 22A, 22B, is constant, and is, for example, between 3 mm and 10 mm.

Advantageously, the first partition 22 comprises a central part 28 able to define a receiving housing 29 for the medical instrument 12.

For example, the central part 28 has a cross-section, in other words, in the plane YZ, in the shape of a semicircle, for example with a diameter between 10 cm and 30 cm.

For example, the receiving housing 29 is delimited by the external face 22B of the central part 28 of the first partition 22.

In particular, the receiving housing 29 is for example, in the shape of a semi-cylinder extending according to the longitudinal axis X.

Alternatively, as in illustrated alternatives, the central part 28 has a cross-section in a U shape.

In one particular embodiment, the first partition 22 also comprises two lateral parts 30 connected to one another by the central part 28, notably according to the transverse axis Y.

In particular, each of the lateral parts 30 of the first partition 22 extends between the upper edge of one of the longitudinal walls 24 and the central part 28.

Preferably, each of the lateral parts 30 extends parallel to the bottom 26.

The first partition 22 is preferably transparent to UV radiation, and notably to UV-C radiation.

In particular, the first partition 22 has a UV transmittance greater than 30%, and preferably greater than 50%, and even more preferably greater than 90%.

By "transmittance", it is meant the ratio of radiation transmitted by the first partition 22 to the radiation incident on the first partition 22.

In other words, at least 30% of the UV radiation incident on the first partition 22, and in particular on its internal face 22A, is transmitted through the first partition 22, and, in particular, exits by the external face 22B.

The first partition 22 is preferably made of quartz, preferably synthetic quartz, such as for example an optical quartz glass of category JGS1.

Alternatively, the first partition 22 is made of a polymer, preferably a cyclic olefin copolymer (COC), in particular TOPAS® 8007X10, or a polymethylpentene, such as TPX^{TM} RT18, or fluorinated ethylene propylene.

The second casing 18 defines a second housing 32 closed by a second partition 34.

Preferably, the second casing 18 comprises at least one wall 36, 37, 38.

For example, the second casing 18 comprises a plurality of walls 36, 37, 38 defining between them the second housing 32.

In the embodiment of the invention illustrated in FIGS. 1 and 2, the plurality of walls of the second casing 18 comprise, for example two longitudinal walls 36, two transverse walls 37 each connecting the two first longitudinal walls 36, and a ceiling 38.

When the enclosure 10 is in the closed position (FIG. 2), the longitudinal walls 36 of the second casing 18 extend, for example, perpendicular to the transverse axis Y, the two transverse walls 37 extending perpendicular to the longitudinal axis X and the ceiling 38 extending perpendicular to the vertical axis Z.

Each of the walls 36, 37, 38 of the second casing 18 is for example a plate, notably substantially rectangular.

In one alternative, not illustrated, the second casing 18 comprises a single wall delimiting the second housing 32, which is for example semi-cylindrical or semi-spherical.

The second partition 34 closes the second housing 32.

In other words, the second housing 32 is a closed volume.

More precisely, the second housing 32 is delimited between the wall or walls 36, 37, 38 of the second casing 18 and the second partition 34.

As is visible in FIG. 2, the second housing 32 is preferably symmetrical to the first housing 20 according to the plane XY, when the enclosure 10 is in the closed position.

In the particular embodiment illustrated, the second partition 34 extends notably facing the ceiling 38 of the second casing 18.

Preferably, the second partition 34 is fixed to each of the two longitudinal walls 36 and the two transverse walls 37 of the second casing 18, and notably on the lower edge of each of these four walls 36,37 as is visible in the Figures.

By lower edge, it is meant here the edge of each of these four walls 36,37 opposite the ceiling 38.

As is visible in the Figures, the second partition 34 comprises an internal face 34A, in particular, oriented toward the second housing 32, and an external face 34B. The two faces 34A, 34B are for example opposite according to the vertical axis Z, when the enclosure 10 is in the closed position.

The second partition 34 is preferably a plate. In particular, the thickness of the second partition 34, in other words, the distance between the two faces 34A, 34B, is constant, and for example between 3 mm and 10 mm.

As is visible in the Figures, the second partition 34 is preferably symmetrical to the first partition 22 according to the plane XY, when the enclosure 10 is in the closed position.

Advantageously, the second partition 34 comprises a central portion 40 able to define a receiving housing 41 for the medical instrument 12.

For example, the central part 40 has a cross-section in the shape of a semicircle, for example with a diameter between 10 cm and 30 cm.

For example, the receiving housing 41 is delimited by the external face 34B of the central part 40 of the second partition 34.

In particular, the receiving housing 41 is for example in the shape of a semi-cylinder extending according to the longitudinal axis X, in the closed position.

Alternatively, in non-illustrated alternatives, the central part 40 of the second partition 34 has a cross-section in a U shape or a rectangular shape.

In one particular embodiment, the second partition 34 also comprises two lateral parts 42 connected to one another by the central part 40.

In particular, each of the lateral parts 42 of the second partition 34 extends between the lower edge of one of the longitudinal walls 36 of the second casing 18 and the central part 40 of the second partition 34.

Preferably, each of the lateral parts 42 of the second partition 34 extends parallel to the ceiling 38 of the second casing 18.

The second partition 34 is preferably transparent to UV radiation, and notably to UV-C radiation.

In particular, the second partition 34 has a UV transmittance greater than 30%, and preferably greater than 50%, and even more preferably greater than 90%.

The second partition 34 is preferably made of quartz, preferably synthetic quartz, such as for example an optical quartz glass of category JGS1.

Alternatively, the second partition 34 is made of a polymer, preferably a cyclic olefin copolymer (COC), in particular TOPAS® 8007X10, or a polymethylpentene, such as TPX^{T}M RT18, or fluorinated ethylene propylene.

As is visible in FIG. 1, in the open position, the first partition 22 and the second partition 34 are for example spaced apart from one another.

More precisely, in the open position, the central parts 28, 40 of the two partitions 22, 34 are spaced apart from one another, in particular so that the two receiving housings 29, 41 do not touch.

As is visible in FIG. 2, in the closed position, the first 22 and the second partition 34 define between them a closed disinfection volume 44.

More precisely, in the closed position, the two partitions 22, 34 extend facing one another.

For example, the two central parts 28,40 extend facing one another, so as to define between them the closed disinfection volume 44, as is visible in FIG. 2.

In the particular illustrated embodiment, the lateral parts 30, 42 of the two partitions 22, 34 extend against one another in the closed position of the enclosure 10.

In particular, the disinfection volume 44 is composed, for example, of the two receiving housings 29, 41.

For example, as is visible in the Figures, the disinfection volume 44 is cylindrical.

Alternatively, in one alternative, not illustrated, in which only one of the two partitions 22, 34 comprises a central part defining a receiving housing, the disinfection volume is constituted only by this single receiving housing.

Preferably, the disinfection volume 44 is configured to adapt to the varied dimensions and shapes of the medical instrument 12.

In an advantageous manner, the disinfection volume 44 is configured so as to be large enough to receive the entire medical instrument 12.

The disinfection volume 44 has, for example, a volume between 3000 cm³ and 100000 cm³.

The enclosure 10 further comprises a UV radiation generation device 46 configured to generate UV radiation, and notably UV-C radiation, toward the disinfection volume 44.

By UV-C radiation, it is meant radiation characterized by a wavelength between 200 and 280 nm.

The UV radiation generation device 46 is arranged in at least one of the first housing 20 and the second housing 32.

In other words, the UV radiation generation device 46 is not arranged inside the disinfection volume 44.

In one particular embodiment, the UV radiation generation device 46 comprises a plurality of UV sources 48 arranged in at least one of the first housing 20 and the second housing 32.

Each source 48 is for example an LED lamp (Light-Emitting Diode).

Advantageously, UV sources 48 are arranged both in the first housing 20 and in the second housing 32.

Preferably, as is visible in FIG. 2, the sources 48 are arranged facing the central part 28, 40 of at least one of the partitions 22, 34.

Even more preferably, the sources 48 are arranged facing the central part 28, 40 of each of the two partitions 22, 34.

In an advantageous manner, the sources 48 are distributed uniformly all around the disinfection volume 44, notably so as to optimize the distribution of UV radiation and thus allow uniform disinfection of the medical instrument 12 arranged in the disinfection volume 44.

Optionally, the first casing 16 also comprises, for example, at least one additional electronic component 50, 52 arranged in the first housing 20.

In particular, the at least one additional electronic component is, for example, an identification unit 50 or a disinfection control unit 52.

The identification unit 50 is, for example, configured to identify the medical instrument 12 to be disinfected, notably to ensure traceability of the disinfection.

By "identify", it is meant more particularly the reading of identification information of the medical instrument 12, such as the identification number of the medical instrument 12 and/or the model of the medical instrument 12.

For example, the identification unit 50 comprises a radio-identification reader configured to read the identification information from an identification tag fixed to the medical instrument 12. The identification tag comprises, for example, a radio-identification chip, also called an RFID chip, containing this identification information. The RFID chip is, for example, a low- frequency chip, a high-frequency chip, for example of the NFC type (Near-Field Communication), or a UHF chip (Ultra High Frequency).

The disinfection control unit 52 is, for example, a processor able to control the disinfection method by the disinfection enclosure.

For example, the disinfection control unit 52 is an electronic circuit designed to manipulate and/or transform data represented by electronic or physical quantities in the processor registers and/or memories into other similar data corresponding to physical data in register memories or other types of display devices, transmission devices or storage devices.

As specific examples, the disinfection control unit 52 is realized in the form of a programmable logic component, such as an FPGA (Field Programmable Gate Array) ,or such as an integrated circuit,such as an ASIC (Application Specific Integrated Circuit) .

As an alternative, when the method is realized in the form of one or more pieces of software, in other words, in the form of a computer program, also called a computer program product, it is further able to be recorded on a medium, not represented, readable by computer. The computer readable medium is, for example, a medium able to store electronic instructions and to be connected to a bus of a computer system. By way of example, the readable medium is an optical disk, a magneto-optical disk, a ROM memory, a RAM memory, any type of non- volatile memory (for example FLASH or NVRAM) or a magnetic card. A computer program comprising software instructions is then stored on the readable medium.

More precisely, the disinfection control unit 52 is configured to control the UV radiation generation device 46, and for example to control each of the sources 48.

For example, the disinfection control unit 52 is configured to implement different disinfection cycles, corresponding for example to different doses and/or durations.

In particular, the disinfection control unit 52 is configured to implement at least one high- level disinfection cycle and a sterilization cycle.

By high-level disinfection, commonly called High-Level Disinfection (HLD), it is meant here a disinfection implying that all viable microorganisms must be killed, with the exception of a small number of spores.

Optionally, the enclosure 10 further comprises a control panel 54, in particular, connected to the disinfection control unit 52, and preferably arranged on the first casing 16.

The control panel 54 comprises, for example, a screen and/or control means, such as buttons for example.

The control panel 54 allows, for example, to choose the disinfection cycle and/or to ensure monitoring of the disinfection.

In addition, the enclosure 10 also comprises, for example, an electrical power supply means 56.

The electrical power supply means 56 comprises, for example, a battery or a power cable.

A method for disinfecting the medical instrument 12 in the enclosure 10 for disinfecting will now be described.

First, an enclosure 10 for disinfecting as described above is provided.

Second, the enclosure 10 for disinfecting is moved to the open position.

Next, the medical instrument 12 intended to be disinfected is positioned in the enclosure 10 for disinfecting.

More precisely, the medical instrument 12 is placed on one of the partitions 22, 34, and, in particular, on the external face 22B, 34B of one of the partitions 22, 34, and notably on one of the central parts 28, 40, and more precisely in one of the receiving housings 29, 41.

For example, the medical instrument 12 is placed on the external face 22B of the central part 28 of the first partition 22, as is visible in FIG. 2.

Preferably, the medical instrument 12 is identified by the identification unit 50.

The enclosure 10 for disinfecting is then moved to the closed position.

More precisely, the second casing 18 is displaced toward its closed position, so as to notably close the disinfection volume 44.

A UV radiation disinfection cycle generated by the UV radiation generation device 46 is then implemented, notably by the disinfection control unit 52.

Such an enclosure 10 for disinfecting is particularly advantageous for disinfecting medical instruments 12, and, in particular, medical probes.

Indeed, thanks to the partitions 22, 34 serving as a support for the medical instrument 12, such an enclosure 10 allows to disinfect a wide range of instruments, of varied sizes and shapes, whether or not they are equipped with cables.

In addition, the partitions 22, 34 prevent the medical instrument from touching the walls of the enclosure 10, which allows to limit the creation of non-disinfected areas (also called cold spots).

Furthermore, the partitions 22, 34 play a barrier role between the UV sources and the medical instrument, which allows to avoid damaging them.

The person skilled in the art will understand that the embodiments and alternatives described previously may be combined with each other, provided that they are technically compatible.

1. An enclosure for disinfecting a medical instrument, the enclosure comprising:
a first casing defining a first housing closed by a first partition, and
a second casing defining a second housing closed by a second partition;
the second casing being movable relative to the first casing between an open position and a closed position,
in the closed position, the first partition and the second partition defining between them a closed disinfection volume;
the enclosure further comprising a UV radiation generation device configured to generate UV radiation in the disinfection volume, the UV radiation generation device being arranged in at least one of the first housing and the second housing.

2. The enclosure according to claim 1, wherein at least one of the first partition and the second partition comprises a central portion able to define a receiving housing for the medical instrument.

3. The enclosure according to claim 1, wherein at least one of the first and the second partition is transparent to UV radiation.

4. The enclosure according to claim 3, wherein at least one of the first and the second partition has a UV transmittance greater than 30%.

5. The enclosure according to claim 4, wherein at least one of the first and the second partition has a UV transmittance greater than 50%.

6. The enclosure according to claim 1, wherein at least one of the first and the second partition is made of quartz.

7. The enclosure according to claim 1, further comprising a hinge arranged between the first casing and the second casing and allowing the second casing to move relative to the first casing.

8. The enclosure according to claim 1, wherein the disinfection volume is cylindrical.

9. The enclosure according to claim 1, wherein the UV radiation generation device comprises a plurality of UV sources arranged in at least one of the first housing and the second housing.

10. The enclosure according to claim 1, wherein the first casing comprises at least one additional electronic component arranged in the first housing.

11. The enclosure according to claim 10, wherein, wherein the at least one electronic component is an identification unit or a disinfection control unit.
